(19) United States
(12) Patent Application Publication
(10) Pub. No.: US 2009/0112149 A1
(43) Pub. Date: Apr. 30, 2009

(54) Variable rate fluid dispenser

(76) Inventors: Marshall S. Kriesel, Saint Paul, MN (US); Joshua W. Kriesel, San Francisco, CA (US)

Correspondence Address: JAMES E. BRUNTON, ESQ., P. O. BOX 29000, GLENDALE, CA, 91209, US

(21) Appl. No.: 11/982,523

(22) Filed: Oct. 31, 2007

(51) Int. Cl. A61M 5/00; B65D 35/00; B65D 35/02; B67D 5/06; B65D 35/40

(52) U.S. Cl. 604/19; 222/95; 222/105; 222/153.01

(57) ABSTRACT

A compact, low-profile fluid dispensing device for use in controllably dispensing fluid medicaments, such as, antibiotics, analgesics and like medicinal agents from the novel bellows-type device reservoir. The dispensing device includes a novel actuating mechanism controllably delivering to the patient the medicament contained within the device reservoir.

Further, the device includes a pair of spring members of novel design that are housed within generally oval-shaped housing and function to provide the force necessary to continuously and substantially uniformly expel fluid from the device reservoir.

### BACKGROUND OF THE INVENTION

1. Field of the Invention

The present invention relates generally to fluid dispensing devices. More particularly, the invention concerns an improved apparatus for infusing medicinal agents into an ambulatory patient at specific rates over extended periods of time, which includes a novel actuating mechanism and a unique adjustable flow rate control means for precisely adjustably controlling the rate of fluid flow from the reservoir of the device toward the patient.

2. Discussion of the Prior Art

Many medicinal agents require an intravenous route for administration of the medicament. The delivery device for delivering the medicament, while not an active pharmacologic agent, may enhance the activity of the drug by mediating its therapeutic effectiveness. Certain classes of new pharmacologic agents possess a very narrow range of therapeutic effectiveness; for instance, too small a dose results in no effect, while too great a dose results in toxic reaction.

In the past, prolonged infusion of fluids has generally been accomplished using gravity flow methods, which typically involve the use of intravenous administration sets and the familiar bottle suspended above the patient. Such methods are cumbersome, imprecise and require bed confinement of the patient. Periodic monitoring of the apparatus by the nurse or doctor is required to detect malfunctions of the infusion apparatus. Devices from which liquid is expelled from a relatively thick-walled bladder by internal stresses within the distended bladder are well known in the prior art. Such bladder, or “balloon”-type, devices are described in U.S. Pat. No. 3,469,578, issued to Bierman and in U.S. Pat. No. 4,318,400, issued to Perry.

One of the most versatile and unique fluid delivery apparatus developed in recent years is that developed by one of the present inventors and described in U.S. Pat. No. 5,205,820. The components of this novel fluid delivery apparatus generally include: a base assembly, an elastomeric membrane serving as a stored energy means, fluid flow channels for filling and delivery, flow control means, a cover, and an ullage which comprises a part of the base assembly.

Another prior art patent issued to one of the present applicants, namely U.S. Pat. No. 5,743,879, discloses an injectable medicament dispenser for use in controllably dispensing fluid medicaments such as insulin, anti-infectives, analgesics, oncolylotics, cardiac drugs bio-pharmaceuticals, and the like from a pre-filled container at a uniform rate. The dispenser, which is quite dissimilar in construction and operation from that of the present invention, includes a stored energy source in the form of a compressively deformable, polymeric, elastomeric member that provides the force necessary to controllably discharge the medicament from a pre-filled container which is housed within the body of the device. After having been deformed, the polymeric, elastomeric member will return to its starting configuration in a highly predictable manner.

Another important prior art fluid delivery device is described in the U.S. Pat. No. 6,063,059 also issued to one of the present inventors. This device, while being of a completely different construction embodies a compressible-expandable stored energy source somewhat similar to that used in the apparatus of the present invention.

As will be appreciated from the discussion which follows, the apparatus of the present invention is uniquely suited to provide precise, continuous fluid delivery management at a low cost in those cases where a variety of precise dosage schemes are of utmost importance. An important aspect of the apparatus of the present invention is the provision of a novel, rotatable fluid flow rate control means that includes uniquely formed micro-capillary, multi-channel flow rate control channels which enable precise control of the rate of fluid flow of the medicament to the patient. More particularly, the apparatus of the present invention includes a novel, adjustable fluid flow rate mechanism which enables the fluid contained within the reservoir of the device to be precisely dispensed at various selected rates.

The apparatus of the present invention can be used with minimal professional assistance in an alternate health care environment such as the home. By way of example, devices of the invention can be comfortably and conveniently, removably affixed to the patient's body or clothing and can be used for the continuous infusion of antibiotics, such as, for example, an antibiotic sold by Abbott Laboratories under the name and style ANCIF and by Rosche under the name and style ROCEPHIN, analgesics such as morphine and like medicinal agents.

By way of summary, the apparatus of the present invention uniquely overcomes the drawbacks of the prior art by providing a novel, disposable dispenser of simple but highly reliable construction. A particularly important aspect of the apparatus of the present invention resides in the provision of a novel, self-contained energy source in the form of a pair of compressible-expandable spring members that provide the force necessary to substantially, uniformly dispense various solutions from the device reservoir. Because of the simplicity of construction of the apparatus of the invention, and the straightforward nature of the energy source, the apparatus can be manufactured at low cost without in any way sacrificing accuracy and reliability.

### SUMMARY OF THE INVENTION

It is an object of the present invention to provide a compact, low-profile fluid dispensing device for use in controllably dispensing fluid medicaments, such as, antibiotics, analgesics and like medicinal agents from the novel bellows-type device reservoir.

It is another object of the invention to provide a fluid dispenser of the aforementioned character which is highly reliable and is easy-to-use by laypersons in a non-hospital environment.

Another object of the invention is to provide a small, compact fluid dispenser that includes a novel actuating mechanism controllably delivering to the patient the medicament contained within the device reservoir.

Another object of the invention is to provide an apparatus which can be factory pre-filled with a wide variety of medicinal fluids that can be easily dispensed in the field.

Another object of the invention is to provide a variable rate fluid dispenser having a low profile housing that is generally oval-shaped in cross section.

Another object of the invention is to provide a variable rate fluid dispenser of the character described in the preceding paragraph in which a stored energy source is provided in the form of a pair of spring members of novel design that are housed within the housing and function to provide the force necessary to continuously and substantially uniformly expel fluid from the device reservoir.

Another object of the invention is to provide a device of the aforementioned character which includes novel adjustable flow rate control means disposed intermediate the fluid reservoir outlet and the outlet port of the device for precisely controlling the rate of fluid flow from the outlet port toward the patient.

Another object of the invention is to provide a fluid dispenser of the class described which is compact, is lightweight, is easy for ambulatory patients to use, is fully disposable, and is extremely accurate so as to enable the infusion of precise doses of medicament over prescribed periods of time.

Another object of the invention is to provide a device of the character described which embodies a novel, easy-to-use disabling mechanism.

Another object of the invention is to provide a self-contained medicament dispenser which is of very simple construction and yet extremely reliable in use.

Another object of the invention is to provide a fluid dispenser as described in the preceding paragraphs which is easy and inexpensive to manufacture in large quantities.

### BRIEF DESCRIPTION OF THE DRAWINGS

FIG. 1 is a generally perspective view of one form of the fluid dispenser of the present invention.

FIG. 2 is a longitudinal, cross-sectional view of the fluid-dispensing device shown in FIG. 1.

FIG. 2A is a greatly enlarged, fragmentary, cross-sectional view of the area designated as “2A” in FIG. 2.

FIG. 3 is a generally perspective, exploded view of the upper portion of the fluid dispenser shown in FIG. 1.

FIG. 4 is a generally perspective, exploded view of the lower portion of the fluid dispenser shown in FIG. 1.

FIG. 5 is a longitudinal, cross-sectional view similar to FIG. 2 but showing the device as it appears following the fluid dispensing step.

FIG. 6 is a top plan view of the rate control selector cover of the device.

FIG. 7 is a cross-sectional view taken along lines 7-7 of FIG. 6.

FIG. 8 is a view taken along lines 8-8 of FIG. 7.

FIG. 9 is a cross-sectional view taken along lines 9-9 of FIG. 8.

FIG. 10 is a top plan view of the rate control selector of the device.

FIG. 11 is a cross-sectional view taken along lines 11-11 of FIG. 10.

FIG. 12 is a view taken along lines 12-12 of FIG. 11.

FIG. 13 is a top plan view of the rate control pick-up member of the device.

FIG. 14 is a cross-sectional view taken along lines 14-14 of FIG. 13.

FIG. 15 is a view taken along lines 15-15 of FIG. 14.

FIG. 16 is a top plan view of the of the rate control housing of the device.

FIG. 17 is a cross-sectional view taken along lines 17-17 of FIG. 16.

FIG. 18 is a view taken along lines 18-18 of FIG. 17.

FIG. 19 is a top plan view of the connector assembly of the device that houses the device reservoir.

FIG. 20 is a cross-sectional view taken along lines 20-20 of FIG. 19.

FIG. 21 is a generally perspective view of the device housing of fluid dispenser of the present invention.

FIG. 22 is a top plan view of the collapsible fluid reservoir of device shown in FIG. 2.

FIG. 23 is a cross-sectional view taken along lines 23-23 of FIG. 22.

FIG. 24 is an exploded, cross-sectional view of the upper portion of the collapsible fluid reservoir shown in FIG. 23.

FIG. 25 is a side elevational view of the rate control assembly of the device shown in FIG. 2.

FIG. 26 is a top plan view of the rate control assembly.

FIG. 27 is a bottom plan view of the rate control assembly.

FIG. 28 is a side elevational view of the upper rate control plate of the rate control assembly of the device shown in FIG. 27.

FIG. 29 is a top plan view of the upper rate control plate of the rate control assembly.

FIG. 30 is a bottom plan view of the upper rate control plate of the rate control assembly.

FIG. 31 is a side elevational view of the lower rate control plate of the rate control assembly of the device shown in FIG. 27.

FIG. 31A is a top plan view of the lower rate control plate of the rate control assembly.

FIG. 32 is a fragmentary, top plan view of the upper portion of the main housing showing the configuration of the locking element that prevents accidental dispensing of the medicinal fluid from the fluid reservoir.

FIG. 33 is a cross-sectional view taken along lines 33-33 of FIG. 32.

FIG. 34 is a cross-sectional view taken along lines 34-34 of FIG. 33.

FIG. 35 is a cross-sectional view taken along lines 35-35 the FIG. 32.

FIG. 36 is a fragmentary, top plan view similar to FIG. 32 but showing the locking member rotated into an unlock, or release position.

FIG. 37 is a cross-sectional view taken along lines 37-37 of FIG. 36.

FIG. 38 is a cross-sectional view taken along lines 38-38 of FIG. 37.

FIG. 39 is a cross-sectional view taken along lines 39-39 of FIG. 37.

FIG. 40 is a fragmentary, cross-sectional view of the lower portion of the dispensing apparatus illustrating the construction of the carriage locking means that prevents movement of the carriage from its first lowered position into its upper, reservoir collapsing position.

FIG. 41 is a cross-sectional view taken along lines 41-41 of FIG. 40.

FIG. 42 is a view taken along lines 42-42 of FIG. 40.

FIG. 43 is a cross-sectional view similar to FIG. 40 but showing the carriage having been released from the rotated locking member.

FIG. 44 as a cross-sectional view taken along lines 44-44 of FIG. 43.

FIG. 45 is a view similar to FIG. 42 but showing the locking member having been rotated into its second release, or unlock position.

FIG. 46 is a cross-sectional view taken along lines 46-46 of FIG. 40.

FIG. 47 is a cross-sectional view taken along lines 47-47 of FIG. 40.

FIG. 48 is a greatly enlarged, fragmentary, cross-sectional view of the area designated in FIG. 47 as “48”.

### DISCUSSION OF THE INVENTION

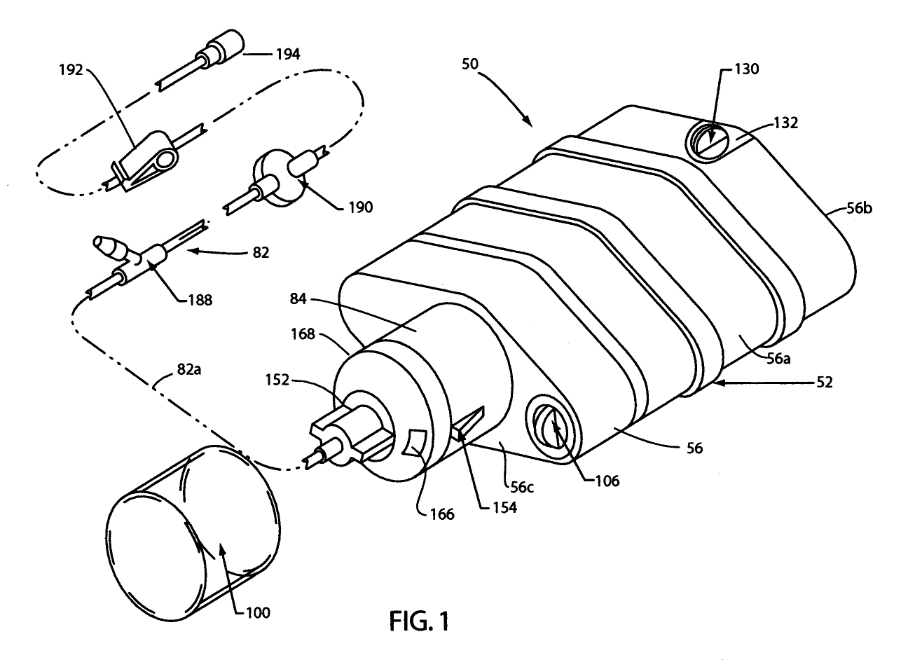

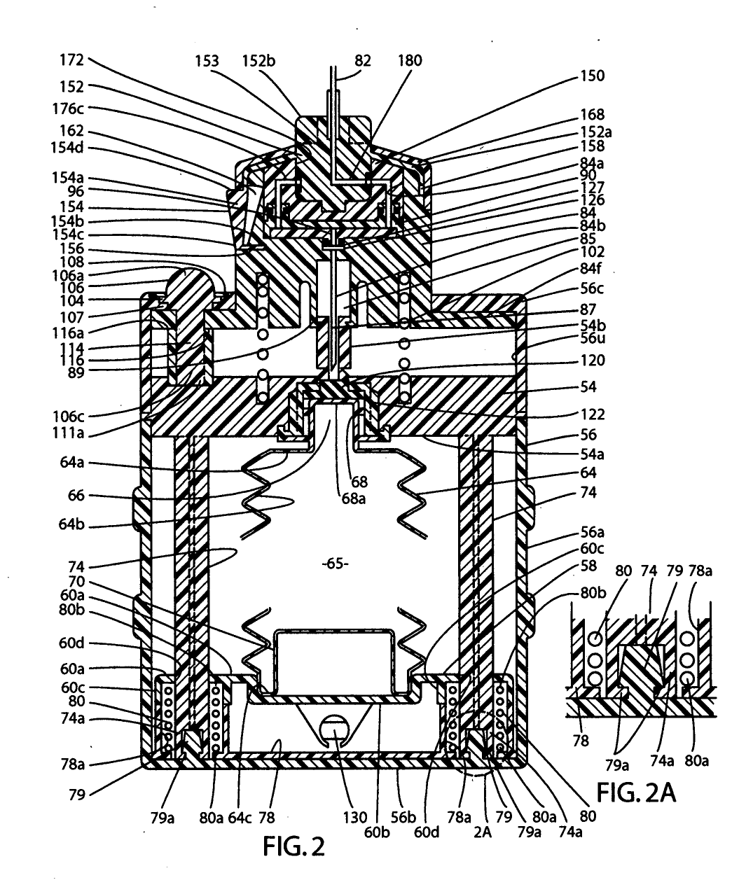

Referring to the drawings and particularly to FIGS. 1 through 5, one form of the dispensing device of the present invention for dispensing medicaments to a patient is there shown and generally designated by the numeral 50. The dispensing device here includes a supporting structure 52, which includes a connector assembly 54 and a main housing 56 that is interconnected with the connector assembly in the manner best seen in FIG. 2 of the drawings. Supporting structure 52 can be constructed from metal, plastic or any suitable material. Main housing 56, which is generally oval-shaped in cross-section, includes a wall portion 56a and a base portion 56b.

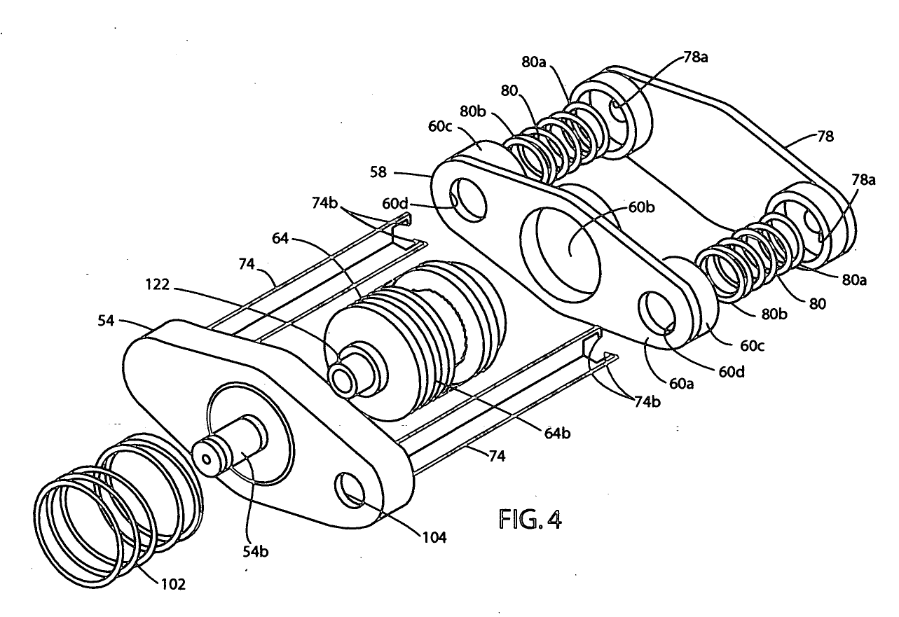

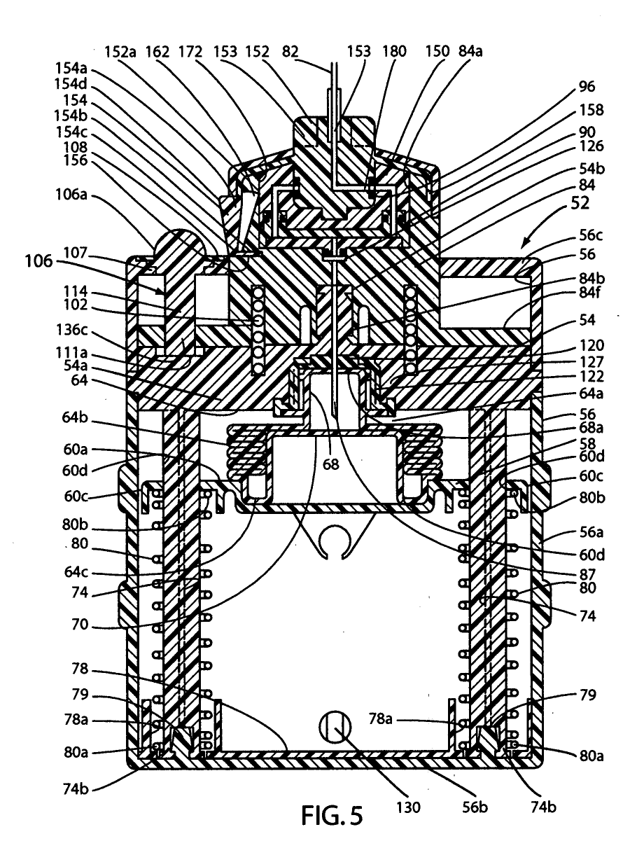

Disposed within wall portion 56a is a carriage 58, which is movable between a first position shown in FIG. 2 and a second position, shown in FIG. 5. As best seen in FIGS. 2 and 4, carriage 58 comprises a generally oval-shaped base plate 60a having a centrally disposed cup-like portion 60b and a pair of transversely spaced, generally cylindrically shaped spring receiving portions 60c. Co-axially aligned with spring receiving portions 60c are a pair of transversely spaced circular bores 60d. Carriage 58 is releasably locked in its first position by a novel locking means the character of which will be described in the paragraphs that follow.

Carried by carriage 58 is a reservoir defining assembly 64 that defines a fluid reservoir 65. As indicated in FIG. 2, reservoir 65 has a combination inlet/outlet 66 that is formed in a neck portion 68. Neck portion 68, which includes a closure wall 68a, is connected to a top wall 64a of the reservoir defining assembly 64, which, in turn, is connected to an accordion-like side wall 64b. Wall 64b is connected to a bottom wall 64c that includes an upstanding ullage portion 70. Top wall 64a, closure wall 68a, side wall 64b and bottom wall 64c cooperate to define the fluid reservoir 65 (see also FIGS. 23 and 24).

In the preferred form of the invention, reservoir defining assembly 64 is constructed in accordance with an aseptic blow-fill technique of a character well understood by those skilled in the art. This aseptic blow-fill technique typically involves the continuous extrusion through an extruder head of a length of parison in the form of a hollow tube between and through two co-acting first or main mold halves. The technique includes the step of cutting off the parison below the extruder head and above the main mold halves to create an opening which allows a blowing and filling nozzle assembly to be moved downwardly into the opening in the parison for molding and thereafter filling a molded container.

When the container is filled with the desired amount of fluid, the blowing and filling nozzle assembly is retracted from the opening in the parison. A separate pair of co-acting second or upper sealing mold halves are then moved together around the exposed length of parison to form and seal the container upper portion. The finished container, completely formed, filled, and sealed, is then conveyed out of the apparatus. Further information concerning aseptic blow-fill techniques is available from Weiler Engineering of Elgin, Ill.

An important feature of the present invention resides in the provision of guide means for guiding travel of carriage 58 between the first position shown in FIG. 2 and the second position shown in FIG. 5. In the present form of the invention this important guide means comprises a pair of transversely spaced guides 74 that extend downwardly from base 54a of connector assembly 54 and are closely received within bores 60d of base plate 60a of carriage 58. As indicated in FIGS. 2 and 5, guides 74 are slidably received within bores 60d so that, as the carriage travels from its first position toward its second position, guides 74 precisely guide its travel.

To controllably move the carriage assembly from its first position to its second position, novel stored energy means are provided. This novel stored energy means, which is operably associated with carriage 58, is here provided in the form of a pair of transversely spaced coiled springs 80. As illustrated in FIGS. 2 and 5, one end 80a of each of the coil springs 80 is disposed in engagement with a generally oval-shaped support plate 78 that is carried by base portion 56b of main housing 56 and is provided with a pair of transversely spaced, generally cylindrically shaped, cup-like spring receiving portions 78a. The other end 80b of each of the coil springs 80 is received within spaced-apart, generally cylindrically shaped, cup-like spring receiving portions 60c of base plate 60a of carriage 58 (see FIG. 4).

As indicated in FIG. 2A, support plate 78 includes a pair of protuberances 79 that are co-axially aligned with spring receiving portions 78a. Turning to FIG. 20, it is to be noted that is the lower portion 74a of each of the guides 74 is provided with a pair of hook-like locking elements 74b that are designed to grip the shoulder portions 79a formed on protuberances 79. In this way, guides 74 are correctly positioned with respect to the carriage so as to enable the carriage to smoothly slide along the guides from its lower position to its upraised position.

With the construction described in the preceding paragraphs, when the fluid reservoir is accessed by the reservoir accessing means of the invention and when the carriage locking means is manipulated in a manner presently to be described to unlock the carriage from the main housing, springs 80 will move from their retracted position shown in FIG. 2 to their expanded position shown in FIG. 5, and in so doing will controllably move the carriage from its starting position shown in FIG. 2 to its fully deployed, or extended, position shown in FIG. 5. As will be described more fully in the paragraphs which follow, as the carriage assembly moves toward its deployed position, the accordion sidewall 64b of the bellows member 64 will move into the collapsed configuration shown in FIG. 5 and in so doing will cause the medicinal fluid contained within the container to be controllably expelled therefrom.

To further control the flow of medicinal fluid from reservoir 65 toward the administration set 82 of the invention (FIG. 1) and then on to the patient, flow control means are provided. This novel fluid flow control means, which is carried by connector assembly 54 of the supporting structure 52, here comprises two cooperating components, namely a rate control means for controlling the rate of fluid flow from the collapsible reservoir toward the administration set and a reservoir accessing means for accessing the collapsible reservoir of the device and for controlling fluid flow between the collapsible reservoir and the rate control means.

The reservoir accessing means, which will be discussed in greater detail hereinafter, here comprises a rate control housing 84, having a chamber 84a and a downwardly extending penetrating member 84b (see FIGS. 2 and 17). Rate control housing 84 is mounted within main housing 56 and is operably associated with the connector assembly 54 in the manner shown in FIGS. 2 and 5. More particularly, housing 84 includes a plurality of locking tabs 85 (FIGS. 17 and 18) that are constructed and arranged to be received within a groove 87 formed in the upstanding neck portion 54b of connector assembly 54 (see also FIG. 20).

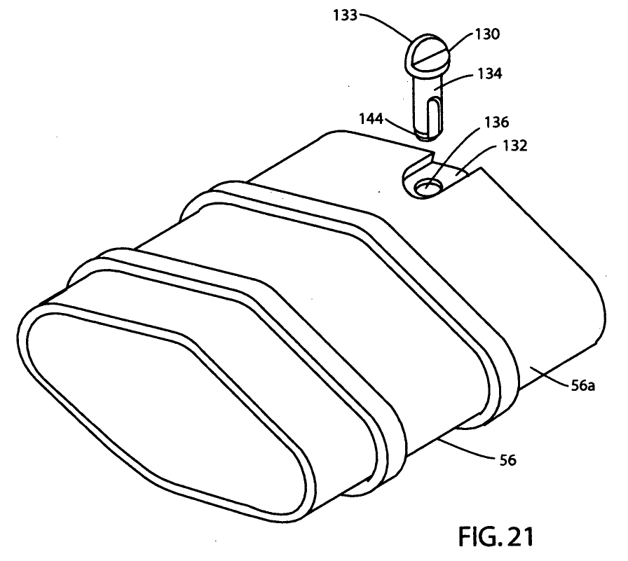

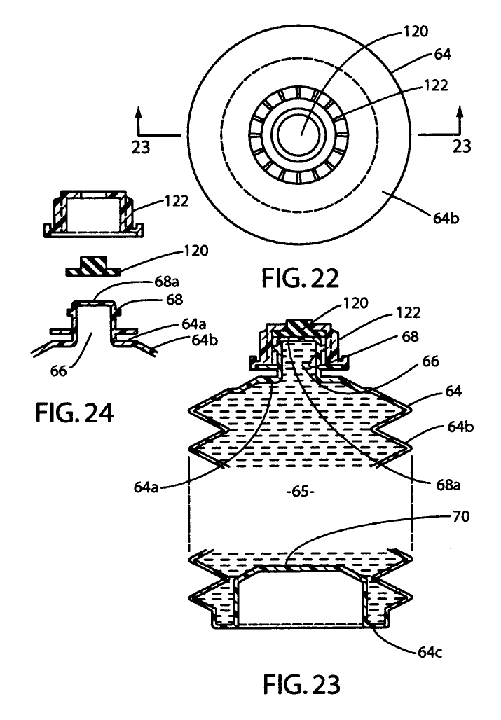

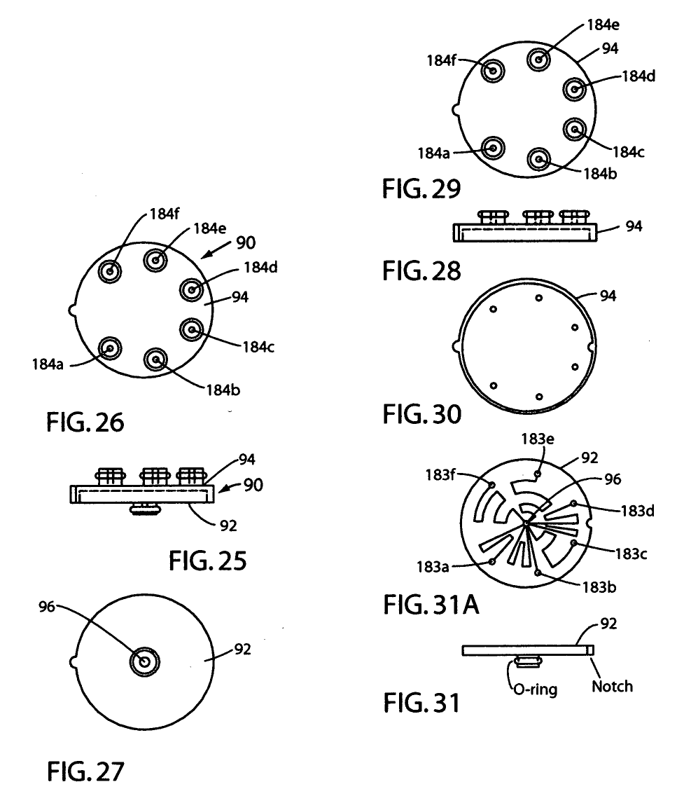

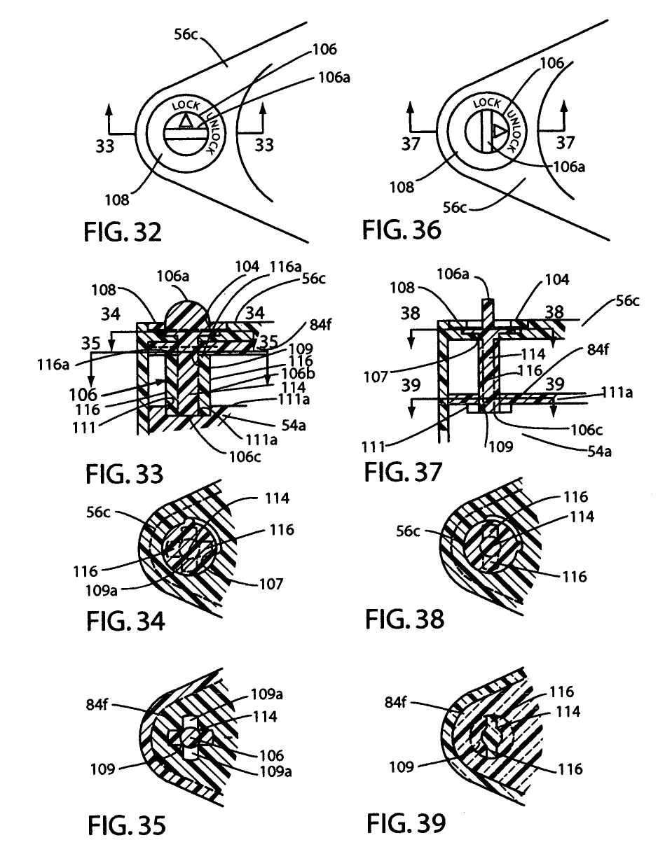

The rate control means of the invention, which is illustrated in FIGS. 22 through 28, includes a rate control assembly 90 that is carried within chamber 84a of the rate control housing 84 in the manner illustrated in FIG. 2. As best seen in FIGS. 25 through 31, rate control assembly 90 here comprises first and second, mating rate control covers 92 and 94 (FIGS. 28 and 31). As will presently be described, rate control cover 92 is provided with a plurality of circuitous fluid flow micro-channels having a central inlet 96 (FIG. 32) that is in fluid communication with outlet of the collapsible reservoir 65 via penetrating member 84b. With the device in the configuration shown in FIG. 2 and with the fluid reservoir 65 filled with the medicament to be dispensed to the patient, the dispensing operation can be commenced by first removing the top cover 100, which is snapped over the upper housing (see FIG. 1). With the cover removed, the administration line 82a of the administration set 82 can be unwrapped from the rate control housing 84 about which it has been coiled. Removal of the top cover 100 exposes the rate control housing 84, which is biased upwardly by a biasing means, shown here as a biasing spring 102 that is supported between rate control housing 84 and the base 54a of the connector assembly 54 in the manner shown in FIG. 2.

Rate control housing 84 is locked against downward movement by the novel rate control locking means of the invention, the operation of which is illustrated in FIGS. 32 through 39. This important locking means, which prevents accidental dispensing of the medicinal fluid here comprises a locking element 106 that is retractably mounted within a cavity 107 formed in the top wall 56c of main housing 56 (FIG. 5). Locking element 106, which is retained within cavity 107 by a locking ring 108, includes a finger grip portion 106a and a shank portion 106b that extends downwardly through a slotted bore 109 formed in the flange portion 84f of rate control housing 84. The lower extremity 106c of the locking element engages a surface 111a formed in a cavity 111 provided in the upper surface of base 54a. The lower extremity 106c of the locking element engages a surface 111a formed in a cavity 111 provided in the upper surface of base 54a of the connector assembly 54 (see FIGS. 2, 5 and 33). Shank portion 106b includes a generally cylindrical central portion 114 and radially outwardly extending, oppositely disposed side walls 116. As indicated in FIG. 33, side walls 116 are slotted to define oppositely disposed locking shoulders 116a that normally engage the lower surface of flange 84f of the rate control housing. However, when the locking element 106 is rotated 90 degrees into the unlock position in the manner shown in FIGS. 36 and 37, locking shoulders 116a move into a position where they can freely pass through the slot portions 109a of slotted bore 109 (see FIGS. 37, 38 and 39). With the locking element 106 rotated into a position where the locking shoulders can freely pass through the slot portions 109a of slotted bore 109 (FIG. 35), a downward force exerted on the rate control housing 84 will cause downward movement of the rate control housing 84 against the urging of spring 102. As the rate control housing 84 moves from the first position shown in FIG. 2 into the second position shown in FIG. 5, the flange portion 84f of housing 84 will slide telescopically through the upper portion 56u of main housing 56.

Downward movement of the rate control housing 84 will cause the penetrating member 84b to first pierce a membrane 120 that is superimposed over closure wall 68a and held in position by a cap 122 (see FIGS. 23 and 24). After piercing membrane 120, the penetrating member will pierce closure wall 68a in the manner shown in FIG. 5. Piercing of the membrane 120 and the closure wall opens a fluid communication path from reservoir 65 to the rate control assembly 90 via a central fluid passageway 87 formed in penetrating member 84b (FIG. 17) and a filter 126 that is disposed within a cavity 127 formed in rate control housing 84.

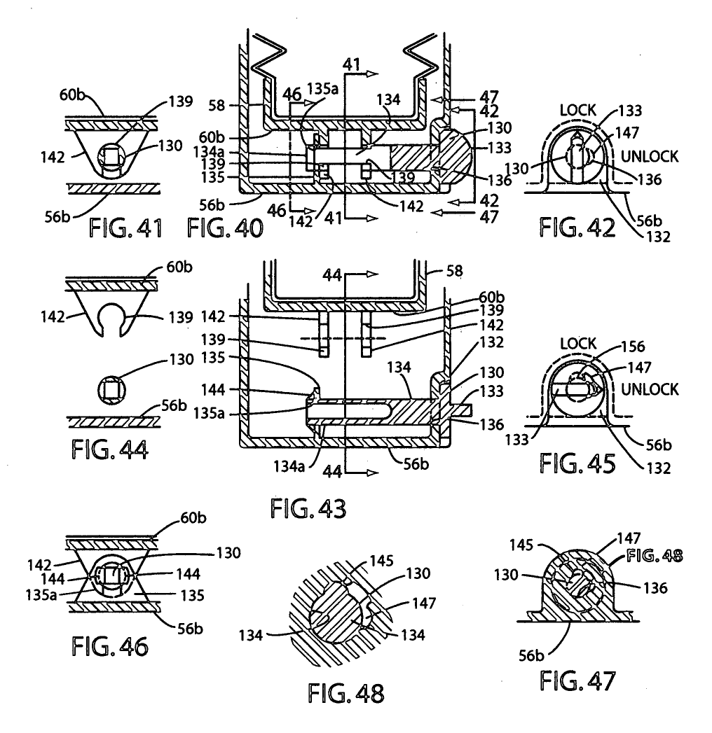

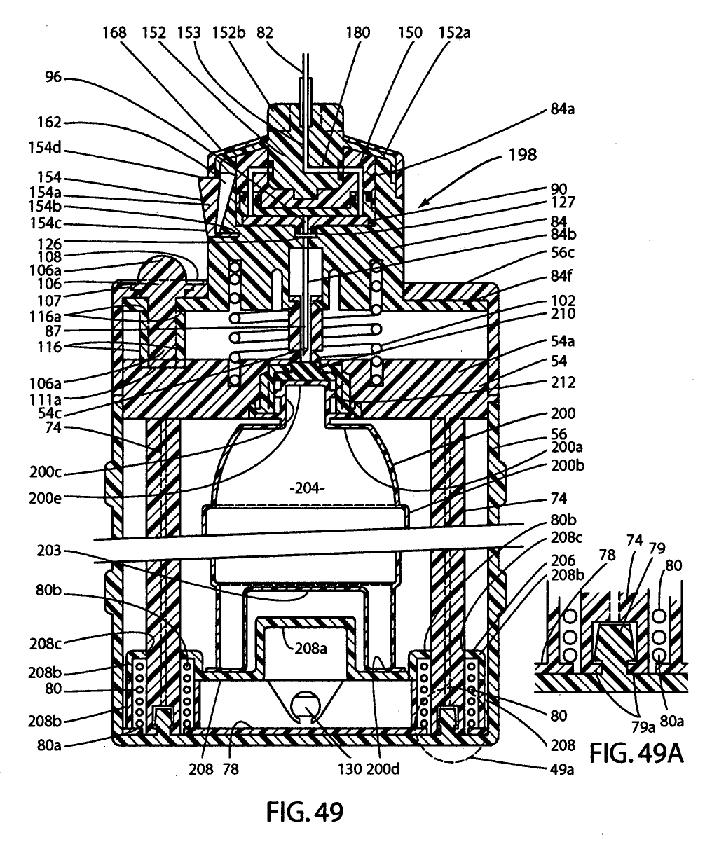

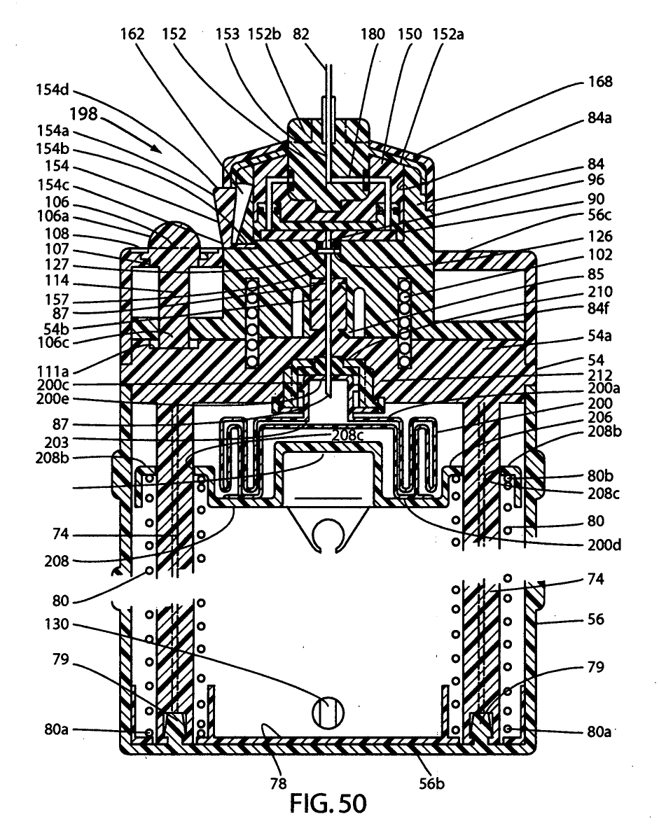

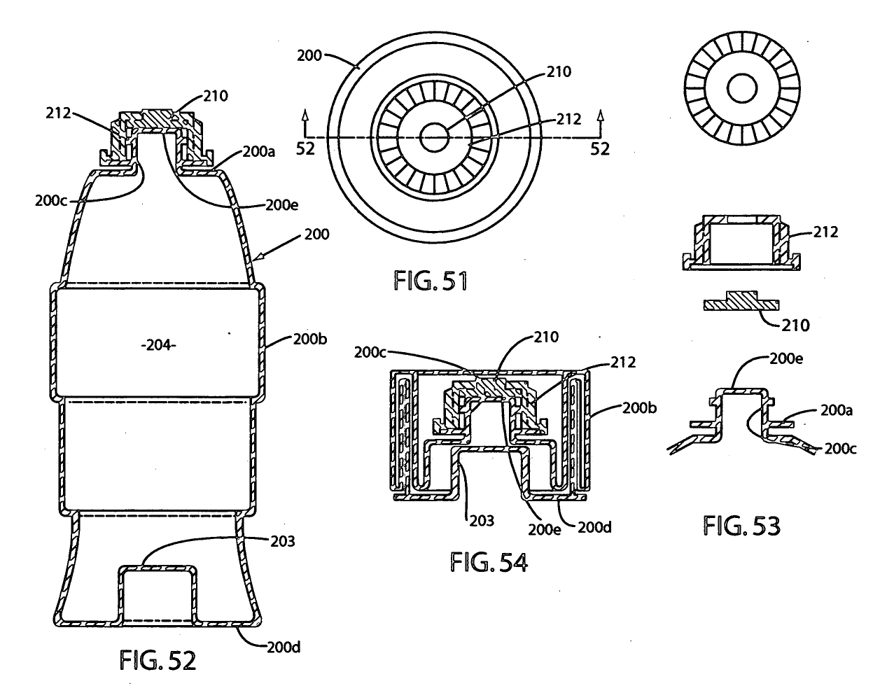

Following release the carriage 58 from the main housing in a manner next to be described, springs 80 will move the carriage from the starting position shown in FIG. 2 to the retracted position shown in FIG. 5. Release of the carriage 58 is accomplished through the manipulation of the novel carriage locking means of the invention. Turning to FIGS. 40 through 48 it can be seen that the carriage locking means here comprises an elongated, transversely extending locking member 130 that is mounted within a recess 132 formed in the lower portion of the main housing (see FIGS. 1 and 43). Locking member 130 includes a finger grip portion 132 and a shank portion 134 that extends inwardly of the main housing through a specially configured bore 136 formed in the lower portion of the main housing (see FIGS. 47 and 48). In the locked position, the inner portion 134a of shank portion 134, which is generally rectangular in cross-section, extends through slotted apertures 139 formed in a pair of spaced-apart locking members 142 that extend downwardly from the carriage base 60a. The inner portion of shank portion 134 also extends through an aperture 135a formed in a locking member 135 that extends upwardly from housing base 56b (FIGS. 43 and 44). As indicated in FIG. 43, radially outwardly extending tabs 144 formed on the inner extremity of shank portion 134 functions to secure the shank portion 134 of the locking member within the locking member 135. With this construction, when the locking member 130 is rotated from the position shown in FIG. 42 the into the position shown in FIGS. 44 and 45, shank portion 134 is free to pass through the slotted aperture 139 of the locking member 142 in the manner illustrated in FIGS. 43 and 44 so that the carriage 58 can move upwardly due to the urging of springs 80. As illustrated in FIGS. 45, 47, 48 and 49, to prevent accidental rotation of locking member 130, the outboard portion of shank 134 is provided with a protuberance 145 that moves through and is guided by a cam-like guide passageway 147 formed in the lower portion of the main housing (see FIG. 48).

As the carriage moves from the starting position to the extended position, the fluid will flow from reservoir 65, through central fluid passageway 84b of penetrating member, through conventional particulate filter 126 carried by housing 84, through inlet 96 of rate control cover 92 and into the circuitous fluid channels formed in the rate control cover 92 (FIG. 31A). In a manner presently to be described in greater detail, from the circuitous fluid channels, the fluid will flow into a selected passageway of the rate control pick-up member 150 (FIG. 2) and toward the administration set 82 via a central passageway 153 formed in rate control selector member 152.

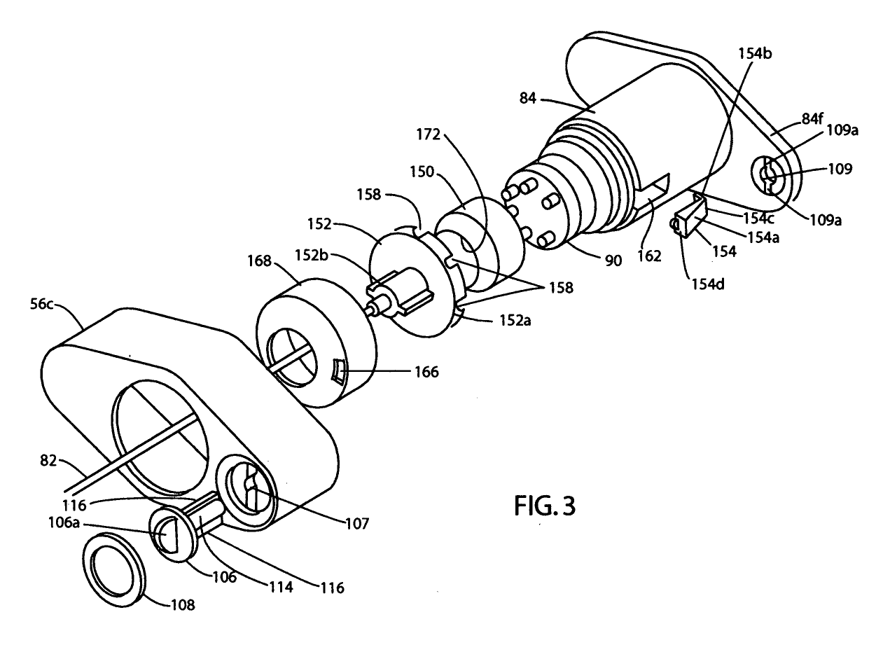

By rotating selector member 152, the rate of fluid flow flowing from reservoir 65 toward the administration set and then on to the patient can be precisely controlled. A unique feature of the apparatus of the invention is the provision of an indexing means that controls the rotation of selector member 152 (see also FIG. 3). This novel indexing means here comprises an indexing latch 154 that is carried by rate control housing 84 for movement between the latched positions shown in FIG. 2 to an inward release position permitting rotation of selector member 152. As best seen in FIG. 3 indexing latch 154 includes a body portion 154a and a tab portion 154b that is interconnected with body portion 54a by a living hinge 154c. Tab portion 154b is closely received within a slot 156 formed in rate control housing 84 and is configured so that the indexing latch 154 is normally biased by the living hinge 154c into the outward latching position shown in FIG. 2. In this outward latching position the shoulder portion 154d of the indexing latch 154 is closely received within a selected one of the circumferentially spaced latching notches 158 (FIGS. 3, 11 and 12) formed in the skirt portion 152a of selector member 152.

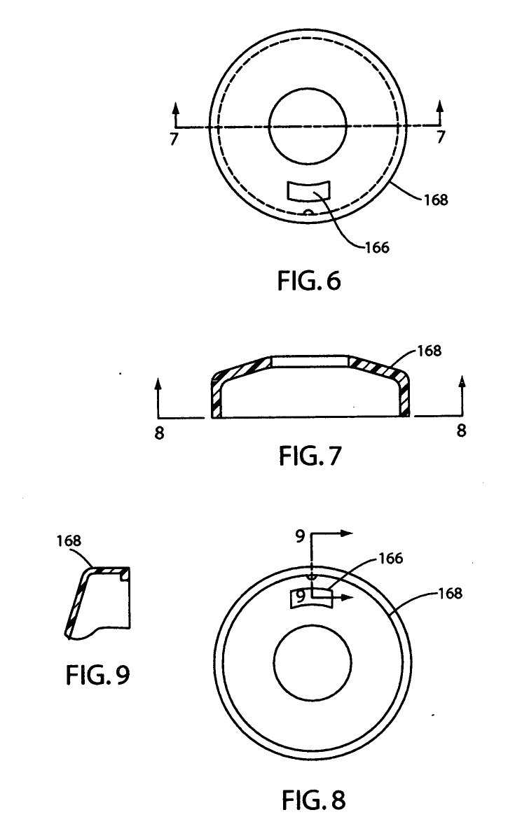

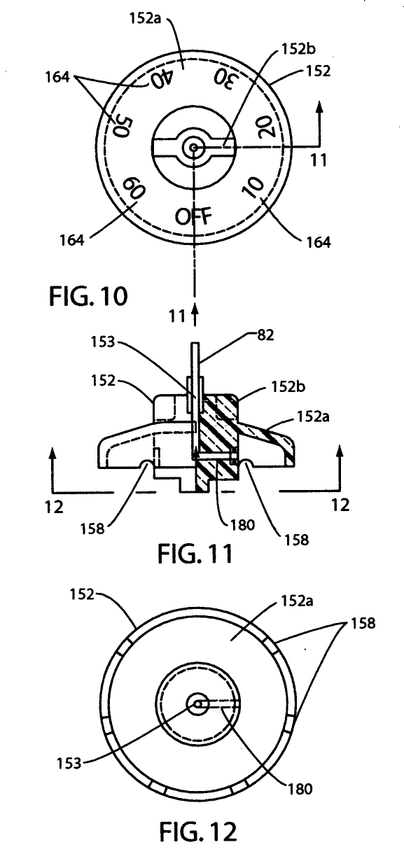

In operation of the latching means, when an inward pressure is exerted on the indexing latch 154 sufficient to move the body portion of the indexing latch into a cavity 162 formed in rate control housing 84 (FIGS. 2 and 3), shoulder 154d will move clear of the latching notch 158 within which it is engaged thereby permitting rotation of the selector member. As the selector member is rotated from the first starting notch toward the second adjacent notch, the living hinge 154c will urge the body potion of the latch into locking engagement with the next adjacent latching notch 158 thereby preventing further rotation of the selector member 152 until an inward pressure is once again exerted on the indexing latch 154 sufficient to move the body portion of the indexing latch into a cavity 162. In the manner presently to be described, this unique construction permits the caregiver to select the precise rate of fluid flow toward the patient. As shown in FIG. 10, indicia 164, which comprise the flow rate control indicia and which are visible through a viewing port 166 formed in the selector member retaining cover 168 (FIG. 6) which is superimposed over the selector member in the manner indicated in FIG. 2 of the drawings.

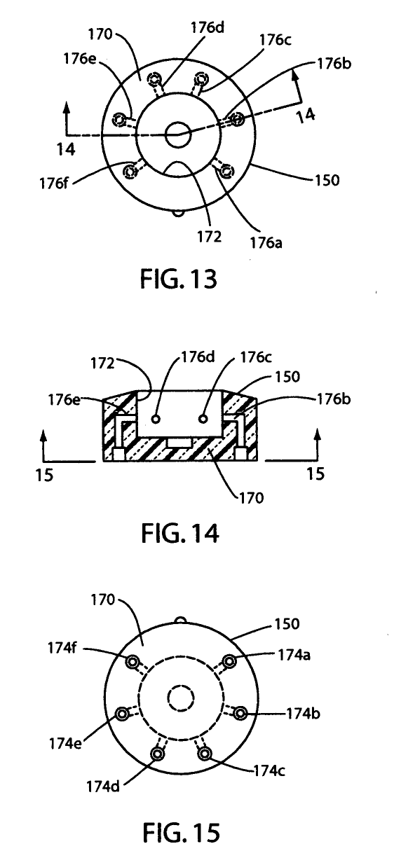

Referring particularly to FIGS. 2 and 13 through 15, the rate control pick-up member 150 of the apparatus of the invention can be seen to have a body portion 170 having a selector member receiving central cavity 172 formed therein. As indicated in FIG. 2, the body portion 152a of the selector member 152 is closely received and is rotatable within central cavity 172 of the rate control pick-up member. Body portion 170 of the rate control pick-up member is provided with circumferentially spaced-apart fluid pick-up ports 174a, 174b, 174c, 174d, 174e and 174f (FIG. 15) that are interconnected with central cavity 172 by fluid passageways 176a, 176b, 176c, 176d, 176e and 176f respectively (FIG. 13). As indicated in FIG. 2, the circumferentially spaced-apart fluid pick-up ports are also in communication with the outlets of the circuitous flow passageways formed in second rate control cover 94 (FIG. 31A). Accordingly, by gripping the finger-engaging portion 152b, the selector member 152 can be rotated in a manner to bring the inlet passageway 180 of the selector member into index with selected one of the circumferentially spaced-apart fluid inlet ports of the rate control pick-up member. In this way fluid communication between inlet passageway 180 and the selected one of the circuitous flow passageways formed in second rate control cover 94 can be established. Since outlet passageway 180 is in fluid communication with the central passageway 153 formed in the rate control member and sinse central passageway 153 is in communication with the administration set 82 of the invention, the rate of fluid flow toward the patient can be precisely controlled by selecting a rate control passageway of appropriate length that is formed in rate control cover 94. As previously mentioned, the indicia 164, which are provided on a flange portion 152a of the selector member and which are viewable through the viewing port and 166 of cover 168, allow the caregiver to precisely select the desired rate of fluid flow toward the patient.

In operating the device of this latest form of the invention, with the fluid reservoir 165 filled with the medicament to be dispensed to the patient, the dispensing operation is commenced by first removing the top cover 100. Next, the indexing means is operated in the manner previously described to permit rotation of the selector member 152 relative to the rate control pick-up member 150 so that the desired rate of fluid flow toward the patient can be selected. In this regard, the caregiver can view the rate control indicia 164 through viewing port 166 in order to select the desired fluid flow rate.

With the desired fluid flow rate thusly selected, the locking means of the invention is operated in the manner previously described to permit downward movement of the penetrating member housing 84 against the urging of spring 102. Downward movement of the rate control housing 94 and the penetrating member housing 84 will cause the penetrating member 84b to first pierce the septal membrane 120 and then the closure wall 68a of the reservoir defining assembly 68 in the manner shown in FIG. 5. Piercing of the membrane 120 and the closure wall opens a fluid communication path from reservoir 65 to the rate control assembly 90 via a central fluid passageway formed in penetrating member 86 and via the filter 127.

Following manipulation of the carriage locking means in the manner previously described to release the carriage 58 from base 56b, the twin spring 80 will move the carriage from the starting position shown in FIG. 2 to the retracted position shown in FIG. 5. As the carriage moves from the starting position to the extended position, the fluid will flow from reservoir 65, through central fluid passageway 85c of penetrating member, through conventional particulate filter 127, through inlet 96 of rate control cover 92 and into the circuitous fluid channels formed in the rate control plate 92 (FIG. 31A). Next the fluid will flow from channel outlets 183a, 183b, 183c, 183d, 183e and 183f (FIG. 30) toward the outlet ports 184a, 184b, 184c, 184d, 184e and 184f of rate control plate 94 (FIGS. 26 and 29). The fluid will then flow into fluid pick-up ports 174a, 174b, 174c, 174d, 174e and 174f of body portion 170 of the rate control pick-up member (FIG. 13). As previously mentioned, controlled rotation of the selector member 152 will move inlet port 158 into register with the selected one of the fluid pick-up ports formed in the rate control pick-up member. From inlet port 158 the fluid will flow into the transverse passageway 180, into central passageway 153 and then into the administration set 82 for controlled delivery to the patient at the selected rate of fluid flow. By way of example, if the caregiver wishes to change the rate of fluid flow toward the patient, the selector member 152 can be controllably rotated from the position shown in FIG. 2 to another position wherein inlet 158 and fluid passageway 180 of the selector member are in communication with a different one of the circumferentially spaced fluid pick-up ports of the rate control pick-up member 150, as for example fluid pick-up port 174e.

In the present form of the invention, administration set 82, which comprises a part of the dispensing means of the invention for delivering medicinal fluids to the patient, includes, in addition to administration line 82a, a conventional “Y”-site injection septum or port 188, a conventional gas vent and particulate filter 190 and a line clamp 192. Provided at the distal end of the administration line is a luer connector 194 of conventional construction (FIG. 1) which enables the device to be interconnected with the patient in a conventional manner.

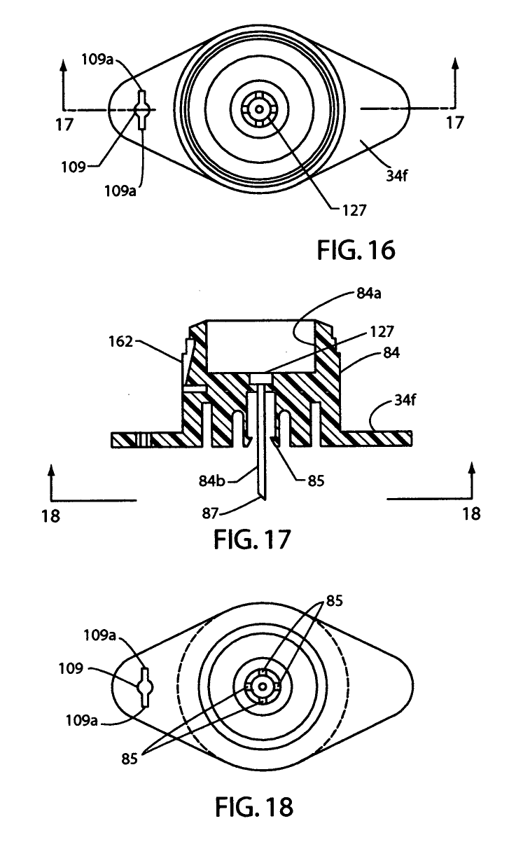

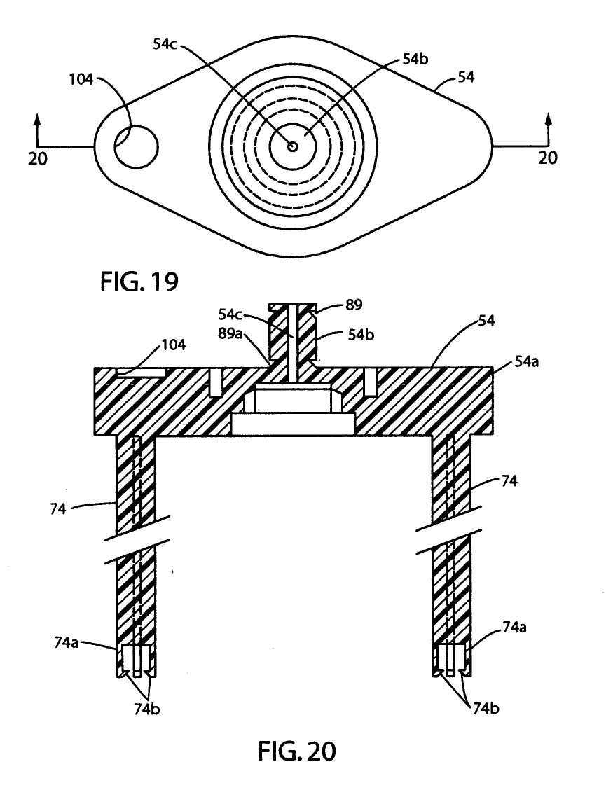

Referring next to FIGS. 49 through 54, yet another form of the dispensing device of the present invention for dispensing medicaments to a patient is there shown and generally designated by the numeral 198. This alternate form of dispensing apparatus is similar in most respects to that shown in FIGS. 1 through 48 and like numerals are used in FIGS. 49 through 54 to identify like components. The major difference between this latest embodiment of the invention and that shown in FIGS. 1 through 48 resides in the differently configured fluid container. As shown in FIGS. 49 and 52, the container, rather than being in the form of a collapsible container, such as container 64, here comprises a collapsible container 200 having a top wall 200a, a collapsible sidewall 200b, a neck 200c and a bottom wall 200d that includes an ullage portion 203. Top wall 200a, collapsible sidewall 200b, neck 200c and bottom wall 200d cooperate to define a fluid reservoir 204 that is connected to the connector member 54 in the manner shown in FIG. 49. In the preferred form of the invention, reservoir 200 is constructed in accordance with the aseptic blow-fill technique of the character previously described.

Container 200 is carried by a carriage 206, which is of somewhat similar construction and operation to carriage 58. More particularly, carriage 206 here comprises a generally oval-shaped base plate 208 having a central collapsible container support portion 208a and a pair of transversely spaced, generally cylindrically shaped spring receiving portions 208b. Co-axially aligned with spring receiving portions 208b are a pair of transversely spaced circular bores 208c. Carriage 206 is releasably locked in its first position by a novel locking means which is substantially identical in construction and operation to that previously described herein.

Guide means of the character previously described, which comprise a pair of transversely spaced guides 74, guide travel of carriage 206 between the first position shown in FIG. 49 and the second position shown in FIG. 50.

To controllably move the carriage assembly from its first position to its second position, novel stored energy means are provided. This novel stored energy means, which comprises a pair of transversely spaced coiled springs 80, is also substantially identical in construction and operation to that previously described herein.

With the construction described in the preceding paragraphs, when the fluid reservoir is accessed by the reservoir accessing means, which is substantially identical in construction and operation to that previously described herein and when the carriage locking means, which is also substantially identical in construction and operation to that previously described herein, is manipulated in a manner to be described to unlock the carriage from the main housing, springs 80 will move from their retracted position shown in FIG. 49 to their expanded position shown in FIG. 50, and in so doing will controllably move the carriage from its starting position to its fully deployed, or extended position shown in FIG. 50. As the carriage assembly moves toward its deployed position, the collapsible sidewall 200b of the container 200 will move into the collapsed configuration shown in FIG. 50 and in so doing will cause the medicinal fluid contained within the container to be controllably expelled therefrom.

To further control the flow of medicinal fluid from reservoir 204 toward the administration set 82 of the invention and then on to the patient, flow control means are provided. This novel fluid flow control means, which is carried by connector assembly 54 of the supporting structure 52, and which comprises two cooperating components, namely a rate control means for controlling the rate of fluid flow from the collapsible reservoir toward the administration set and a reservoir accessing means for accessing the collapsible reservoir of the device and for controlling fluid flow between the collapsible reservoir and the rate control means is also substantially identical in construction and operation to that previously described herein.

With the device in the configuration shown in FIG. 49 and with the fluid reservoir 204 filled with the medicament to be dispensed to the patient, the dispensing operation can be commenced by first removing the top cover 100, which is snapped over the upper housing (see FIG. 1). With the cover removed, the administration line 82a of the administration set 82 can be unwrapped from the rate control housing 84 about which it has been coiled. Removal of the top cover 100 exposes the rate control housing 84, which is biased upwardly by a biasing means, shown here as a biasing spring 102 that is supported between rate control housing 84 and the base 54a of the connector assembly 54 in the manner shown in FIG. 2.

Rate control housing 84 is locked against downward movement by the novel rate control locking means of the invention, the operation of which is illustrated in FIGS. 32 through 39. This rate control locking means, which prevents accidental dispensing of the medicinal fluid is also substantially identical in construction and operation to that previously described herein and here comprises a locking element 106 that is retractably mounted within a cavity 107 formed in the top wall 56c of main housing 56 (FIG. 49).

As in the earlier described embodiment, downward movement of the rate control housing 84 will cause the penetrating member 84b to first pierce a membrane 210 that is superimposed over the closure wall 200d of the collapsible container and held in position by a cap 212 (see FIGS. 52 and 53). After piercing membrane 210, the penetrating member will pierce closure wall 200d in the manner shown in FIG. 50. Piercing of the membrane 210 and the closure wall opens a fluid communication path from reservoir 204 to the rate control assembly 90 via a central fluid passageway 87 formed in penetrating member 84b (FIG. 17) and a filter 126 that is disposed within a cavity 127 formed in rate control housing 84.

Following release of the carriage 206 from the main housing in the manner previously described springs 80 will move the carriage from the starting position shown in FIG. 49 to the retracted position shown in FIG. 50. As before, release of the carriage 206 is accomplished through the manipulation of the novel carriage locking means of the invention which here comprises an elongated, transversely extending locking member 130 that is mounted within a recess 132 formed in the lower portion of the main housing. The novel carriage locking means of this latest form of the invention is also substantially identical in construction and operation to that previously described herein.

Following release of the carriage it will move from the starting position shown in FIG. 49 to the extended position shown in FIG. 50. This movement of the carriage will cause the fluid to flow from reservoir 65, through central fluid passageway 84b of penetrating member, through conventional particulate filter 126 carried by housing 84, through inlet 96 of rate control cover 92 and into the circuitous fluid channels formed in the rate control cover 92 (FIG. 31A). In the manner previously described, from the circuitous fluid channels, the fluid will flow into a selected passageway of the rate control pick-up member 150 (FIG. 2) and toward the administration set 82 via a central passageway 153 formed in rate control selector member 152.

By rotating selector member 152, the rate of fluid flow flowing from reservoir 65 toward the administration set and then on to the patient can be precisely controlled. As before the indexing means of the device, which comprises the indexing latch 154 and which is also substantially identical in construction and operation to that previously described herein, controls the rotation of selector member 152 (see also FIG. 3).

As previously described, by gripping the finger-engaging portion 152b of the selector member 152, the selector member can be rotated in a manner to bring the inlet passageway 180 of the selector member into index with the selected one of the circumferentially spaced-apart fluid inlet ports of the rate control pick-up member. In this way fluid communication between inlet passageway 180 and the selected one of the circuitous flow passageways formed in second rate control cover 94 can be established. Since outlet passageway 180 is in fluid communication with the central passageway 153 formed in the rate control member and since central passageway 153 is in communication with the administration set 82 of the invention, the rate of fluid flow toward the patient can be precisely controlled by selecting a rate control passageway of appropriate length that is formed in rate control cover 94. As previously mentioned, the indicia 164, which are provided on a flange portion 152a of the selector member and which are viewable through the viewing port and 166 of cover 168, allow the caregiver to precisely select the desired rate of fluid flow toward the patient.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. A dispensing apparatus for dispensing medicaments to a patient comprising:
(a) a supporting structure;
(b) a pre-filled collapsible fluid reservoir carried by said supporting structure, said collapsible reservoir having an outlet port comprising sealing means for sealing said outlet port;
(c) stored energy means carried by said supporting structure and operably associated with said collapsible reservoir for collapsing said collapsible reservoir to expel fluid therefrom;
(d) an administration set, including an administration line interconnected with said outlet of said collapsible reservoir; and
(e) fluid flow control means carried by said supporting structure for controlling fluid flow from said collapsible reservoir toward said administration set.

2. The dispensing apparatus as defined in claim 1 in which said sealing means comprises a pierceable member.

3. The dispensing apparatus as defined in claim 1 in which said stored energy means comprises a pair of transversely spaced-apart springs operably interconnected with said collapsible reservoir. a shearable member,

4. The dispensing apparatus as defined in claim 1 in which said pre-filled collapsible fluid reservoir is aseptically filled and sealed at time of manufacture.

5. The dispensing apparatus as defined in claim 1, further comprising a carriage movable between a first position and a second position, said pre-filled collapsible fluid reservoir being carried by said carriage.

6. The dispensing apparatus as defined in claim 1 in which flow control means comprises rate control means for controlling the rate of fluid flow from said collapsible reservoir toward said administration set.

7. The dispensing apparatus as defined in claim 1 in which said flow control means comprises rate control means for controlling the rate of fluid flow from said collapsible reservoir toward said administration set and further comprises operating means for controlling fluid flow between said collapsible reservoir and said rate control means.

8. The dispensing device as defined in claim 7 in which said rate control means includes selector means for selecting the rate of fluid flow between said collapsible reservoir and said administration set.

9. The dispensing device as defined in claim 8 in which said selector means comprises a selector housing carried by said supporting structure and a selector member rotatably carried by said selector housing.

10. A dispensing apparatus for dispensing medicaments to a patient comprising:
(a) a supporting structure comprising a base assembly and a housing interconnected with said base assembly;
(b) a carriage assembly interconnected with said supporting structure for movement between a first position and a second position;
(c) a pre-filled, collapsible reservoir carried by said carriage assembly, said collapsible reservoir having an outlet port;
(d) guide means connected to said supporting structure for guiding travel of said carriage assembly between said first position and said second position, said guide means comprising a guide member connected to said base assembly and a guide rib connected to said housing of said supporting structure;
(e) a stored energy means operably associated with said carriage assembly for moving said carriage assembly between said first and second positions, said stored energy means comprising a pair of spaced-apart springs;
(f) an administration set, including an administration line interconnected with said outlet port of said collapsible reservoir; and
(g) fluid flow control means carried by said base assembly of said supporting structure for controlling fluid flow from said collapsible reservoir toward said administration set.

12. The dispensing apparatus as defined in claim 11, further including locking means carried by said supporting structure for locking said carriage assembly in said first position.

13. The dispensing apparatus as defined in claim 11 in which said collapsible reservoir comprises a bellows structure.

14. The dispensing apparatus as defined in claim 11 in which said collapsible reservoir comprises a collapsible container.

15. The dispensing apparatus as defined in claim 11 in which said flow control means comprises:
(a) rate control means carried by said supporting structure for controlling the rate of fluid flow from said collapsible reservoir toward said administration set; and
(b) operating means carried by said supporting structure for controlling fluid flow between said collapsible reservoir and said rate control means.

16. The dispensing device as defined in claim 15 in which said rate control means includes a rate control housing carried by said supporting structure and a selector member rotatably carried by said rate control housing.

17. The dispensing device as defined in claim 16 in which said rate control means further includes an indexing latch carried by said rate control housing for movement between a latched position and an inward release position permitting rotation of said selector member.

18. A dispensing apparatus for dispensing medicaments to a patient comprising:
(a) a supporting structure comprising a base assembly and a generally oval-shaped outer housing interconnected with said base assembly;
(b) a carriage assembly interconnected with said supporting structure for movement between a first position and a second position, said carriage having a carriage base provided with a pair of spaced-apart openings;
(c) locking means carried by said supporting structure for locking said carriage assembly in said first position;
(d) an aseptically filled collapsible container carried by said carriage assembly, said collapsible container comprising a collapsible side wall, a top wall connected to said collapsible side wall and accessing means sealably connected to said top wall.
(e) guide means connected to said supporting structure for guiding travel of said carriage assembly between said first position and said second position, said guide means comprising a pair of spaced-apart guide members connected to said base assembly, said guide members being slidably received within said openings provided in said carriage base;
(f) a stored energy means operably associated with said carriage assembly for moving said carriage assembly between said first and second positions, said stored energy means comprising a pair of spaced-apart coil springs;
(g) an administration set, including an administration line interconnected with said outlet port of said collapsible reservoir; and
(h) fluid flow control means carried by said base assembly of said supporting structure for controlling fluid flow from said collapsible reservoir toward said administration set, said flow control means comprising:
(i) rate control means carried by said supporting structure for controlling the rate of fluid flow from said collapsible reservoir toward said administration set, said rate control means comprising a rate control plate having a plurality of fluid flow channels interconnected with said outlet of said collapsible reservoir; and
(ii) operating means carried by said supporting structure for controlling fluid flow between said collapsible reservoir and said rate control means.

19. The dispensing apparatus as defined in claim 18 in which said operating means comprises a penetrating member movable between first position and a second position permitting fluid flow from said collapsible reservoir toward said administration set.

20. The dispensing device as defined in claim 19 in which said rate control means further includes selector means for selecting the rate of fluid flow between said collapsible reservoir and said administration set, said selector means comprising a selector housing carried by said supporting structure and a selector member rotatably carried by said selector housing.
